(19) United States
(12) Patent Application Publication
(10) Pub. No.: US 2021/0189229 A1
(43) Pub. Date: Jun. 24, 2021

(54) TRACER FOR USE IN A SUBTERRANEAN WELL

(71) Applicant: RESERVOIR METRICS, LLC, Houston, TX (US)

(72) Inventors: David J. WINTERGRASS, Katy, TX (US); Alexander J. DARNLEY, Houston, TX (US); Christopher DEMPSEY, Houston, TX (US)

(21) Appl. No.: 17/126,341

(22) Filed: Dec. 18, 2020

Related U.S. Application Data
(60) Provisional application No. 62/950,914, filed on Dec. 19, 2019.

(51) Int. Cl. C09K 8/92; C09K 8/60; G01N 33/28; E21B 47/11

(52) U.S. Cl. CPC C09K 8/92; C09K 8/602; C09K 2208/10; E21B 47/11; G01N 33/2882

(57) ABSTRACT

A chemical tracer particle can include a polymer core, a tracer chemical embedded in the polymer core, and an outer layer surrounding the polymer core, the outer layer including a surfactant. A method of tracing flow of well fluid from each of multiple stages in a well can include injecting into each stage a respective one of multiple chemical tracers, each of the chemical tracers including chemical tracer particles, each of the chemical tracer particles including a polymer core, a tracer chemical embedded in the polymer core, and an outer layer surrounding the polymer core, the outer layer including a surfactant, and producing the chemical tracers from the stages. A method of producing a chemical tracer particle can include generating a polymer core having a tracer chemical embedded therein, and surrounding the polymer core with an outer layer including a surfactant.

### CROSS-REFERENCE TO RELATED APPLICATION

This application claims the benefit of the filing date of U.S. provisional application No. 62/950,914 filed on 19 Dec. 2019. The entire disclosure of the prior application is incorporated herein by this reference.

### BACKGROUND

In a typical tracer operation, a known tracer chemical is injected into a conventional or unconventional petroleum well during a hydraulic fracturing operation. When the well is flowed back, samples of well fluids (including, e.g., oil and water) are collected and tested for the tracer chemical. The results are then used as a diagnostic tool for an operator to understand how well the fracturing operation occurred.

It will, therefore, be appreciated that improvements are continually needed in the art of evaluating formations and fractures therein utilizing chemical tracers. Such improvements may be useful whether or not a well has been fractured.

### BRIEF DESCRIPTION OF THE DRAWINGS

FIG. 1 is a representative cross-sectional view of an example of a system and method that can incorporate principles of this disclosure.

FIG. 2 is a representative cross-sectional view of a chemical tracer particle that may be used in the FIG. 1 system and method, and which can incorporate the principles of this disclosure.

### DETAILED DESCRIPTION

Described below is an improved utilization of tracer chemical. In one example, instead of injecting the raw chemical into a formation, the tracer chemical is incorporated into a nanoparticle. The advantages of this include, but are not limited to: the particle size and surface properties can be controlled so all of the nanoparticles will have the same solubilities. Although the tracer chemical which is incorporated may be different for respective different stages into which the chemical tracer is injected, the behavior will be the same for all of the nanoparticles.

In one aspect, a method can include producing a polymer nano bead with a tracer chemical incorporated therein. Different tracer chemicals may be incorporated into respective different polymer nano beads, and these different nano beads can have a same solubility in well fluids.

The novel chemical tracer particle (such as, nanoparticles or nano beads having a tracer chemical incorporated therein) can be used as a diagnostic tool during such operations as hydraulic fracturing and enhanced oil recovery. During a hydraulic fracturing operation, much information can be ascertained by incorporating a chemical tracer particle into one or more individual fracturing stages. For enhanced oil recovery operations, a chemical tracer particle can be injected along with an initial flooding fluid into a field location to yield information about the field location.

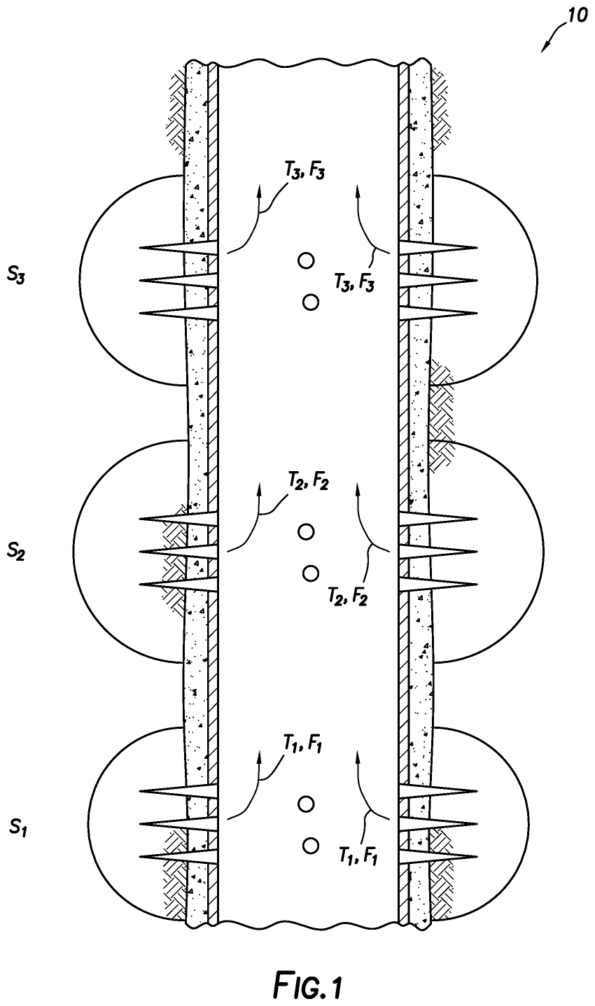

Representatively illustrated in FIG. 1 is a system 10 for use with a subterranean well, and an associated method, which can embody principles of this disclosure. However, it should be clearly understood that the system 10 and method are merely one example of an application of the principles of this disclosure in practice, and a wide variety of other examples are possible. Therefore, the scope of this disclosure is not limited at all to the details of the system 10 and method described herein and/or depicted in the drawings.

In examples described below, chemical tracer particles are synthesized. Some advantages of the chemical tracer particles can include: the solubility properties of the chemical tracer particles can be ultimately controlled by an applied outside layer, a surface chemistry will be similar for all of the chemical tracer particles, a size of the chemical tracer particles can be controlled during the synthesis procedure, a stability or lifetime of the chemical tracer particles is determined from the polymer used, and analytical methods for determining the amount of tracer chemical material can be based on routine chemical identification and quantitation.

These overall characteristics will determine water solubility, partitioning coefficients, and reservoir residence time. The new chemical tracer particles can have uniform properties, so that they will behave the same during a typical application.

With the size and surface chemistry the same, even though the tracer chemical incorporated therein differs, the individual particles will all be affected in the same way when exposed to external physiochemical phenomena such as adsorption in a reservoir. Therefore, even though a unique chemical is incorporated into each chemical tracer particle, the result of their exposure to the reservoir and well fluids will be substantially the same.

The chemical tracer particle comprises a base polymer core with a selected tracer chemical embedded therein, and an applied outer layer of surfactant type molecules. In one example, a nanoparticle polymer core can be manufactured using a micro-fluidics technique, in which the polymer is generated through interfacial polymerization.

Interfacial polymerization consists of contacting an aqueous layer of fluid with an organic layer of fluid. Each layer contains a component of the chosen polymer, with polymerization occurring when the layers are in contact with each other.

The chemical to be used as the tracer material is added to one of the above described layers, depending on its solubility. Various polymers can be created using this method including, but not limited to, polyamides, polyanilines, polyimides, polyurethanes, polyureas, polypyrroles, polyesters, polysulfonamides, polyphenyl esters and polycarbonates.

The tracer chemicals that can be embedded in the polymer matrix include, but are not limited to, the following classes: halogenated benzenes, halogenated aliphatics, halogenated benzoic acids, halogenated benzoic esters, halogenated aldehydes, halogenated ethers, and halogenated alcohols.

Depending on the polymer, various surfactants can be coated onto the surface of the nanoparticles using the micro-fluidic synthesis device. In this example, the outside coating determines the solubility of the nanoparticle in well fluid.

Upon collecting flowback fluid from the field, a sample is analyzed (such as, in a laboratory) for the embedded tracer chemical(s). For aqueous based samples, they are mixed with a solvent, acid or base depending on the polymer material. The tracer chemical is then separated from the polymer and is in solution with the solvent, acid or base.

The tracer chemical material is then extracted and concentrated from this solution. The identity and quantitation of the tracer chemical are completed using, for example, gas chromatography, gas chromatography-mass spectrometry, or liquid chromatography-mass spectrometry.

For oil-based samples, they are mixed with a solvent, acid or base depending on the polymer material. The chemical is then separated and concentrated using known laboratory techniques. The resulting extract is analyzed by, for example, gas chromatography, gas chromatography-mass spectrometry, or liquid chromatography-mass spectrometry.

In the FIG. 1 example, three chemical tracers (comprising chemical tracer particles) T1-3 have been injected into zones or stages S1-3. The stages S1-3 are then produced as depicted in FIG. 1. The tracers T1-3 are flowed back, along with respective fluids F1-3 from each stage S1-3.

If all of the tracers T1-3 have a same size and are surface coated with the same surfactant, they will all have the same solubility in the same well fluids and will be adsorbed the same in the same reservoir rock. Thus, different tracer chemicals can be used in the respective different tracers T1-3, thereby enabling flowback from the different stages S1-3 to be separately identified, even if they are commingled as they are produced from the well.

Note that it is not necessary for the stages S1-3 to be fractured in this method. For example, the tracers T1-3 could be injected into the zones or stages S1-3 from an offset injection well, whether or not the production well is fractured.

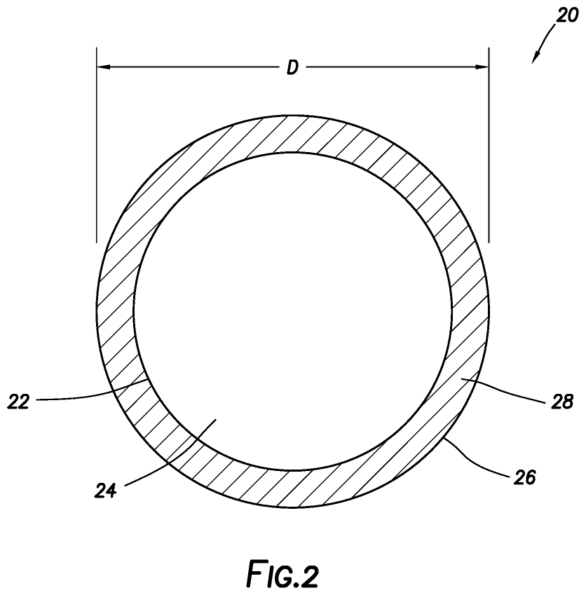

Referring additionally now to FIG. 2, a cross-sectional view of an example of a chemical tracer particle 20 is representatively illustrated. The chemical tracer particle 20 may be used in the system 10 and method of FIG. 1, or it may be used in other systems and methods.

In this example, the particle 20 comprises a polymer matrix or core 22 having a tracer chemical 24 embedded therein. The polymer core 22 can be manufactured using a micro-fluidics technique as described above, in which the polymer is generated through interfacial polymerization.

The polymer core 22 may comprise, for example, a polyamide, polyaniline, polyimide, polyurethane, polyurea, polypyrrole, polyester, polysulfonamide, polyphenyl ester and/or polycarbonate material. The tracer chemical 24 may comprise, for example, a halogenated benzene, halogenated aliphatic, halogenated benzoic acid, halogenated benzoic ester, halogenated aldehyde, halogenated ether and/or halogenated alcohol.

The FIG. 2 particle 20 also comprises a coating or outer layer 26. The outer layer 26 comprises a surfactant 28. Any surfactant suitable for use in well fluids (including fluids produced from or injected into wells, such as, in fracturing or well flood operations, etc.) may be used.

As depicted in FIG. 2, the particle 20 has a diameter or maximum outer dimension D. Preferably, the outer dimension D is a maximum of 1000 nm.

In some examples, the particle 20 may be a “nanoparticle” as that term is understood by those skilled in the art. A nanoparticle is generally considered to be an object having all external dimensions between 1 nm and 1000 nm.

Although the FIG. 2 particle 20 comprises only the core 22 and the outer layer 26, in other examples additional components may be used. For example, an additional layer could be interposed between the core 22 and the outer layer 26.

The core 22 is a “core” in that it is surrounded by the outer layer 26. In other examples, another core or inner layer could be enclosed in the core 22.

It may now be fully appreciated that the above disclosure provides significant advancements to the art of evaluating formations and/or fractures therein utilizing chemical tracers. In one example described above, different tracer chemicals 24 can be incorporated into respective different particles 20, which are then injected into respective different stages S1-3. However, the particles 20 are adsorbed into the different stages S1-3 at a same rate, since they all have a same outer layer 26 comprising a surfactant 28.

The above disclosure provides to the art a chemical tracer particle 20. In one example, the chemical tracer particle 20 can comprise a polymer core 22, a tracer chemical 24 embedded in the polymer core 22, and an outer layer 26 surrounding the polymer core 22. The outer layer 26 comprises a surfactant 28.

The particle 20 may have a maximum outer dimension D less than or equal to 1000 nm. The particle 20 may be a nanoparticle.

The polymer core 22 may comprise a polymer material selected from polyamide, polyaniline, polyimide, polyurethane, polyurea, polypyrrole, polyester, polysulfonamide, polyphenyl ester and polycarbonate.

The tracer chemical 24 may comprise at least one of halogenated benzene, halogenated aliphatic, halogenated benzoic acid, halogenated benzoic ester, halogenated aldehyde, halogenated ether and halogenated alcohol.

A method of tracing flow of well fluid from each of multiple stages S1-3 in a subterranean well is also provided to the art by the above disclosure. In one example, the method can comprise: injecting into each stage S1-3 a respective one of multiple chemical tracers T1-3. Each of the chemical tracers T1-3 can comprise chemical tracer particles 20. Each of the chemical tracer particles 20 can comprise a polymer core 22, a tracer chemical 24 embedded in the polymer core 22, and an outer layer 26 surrounding the polymer core 22. The outer layer 26 can comprise a surfactant 28. The method includes producing the chemical tracers T1-3 from the stages S1-3.

Each of the chemical tracers T1-3 may comprise a respective different one of the tracer chemicals 24.

All of the chemical tracers T1-3 may comprise the same surfactant 28.

The chemical tracers T1-3 may be adsorbed at a same rate in all of the stages S1-3.

A method of producing a chemical tracer particle 20 is also described above. In one example, the method can comprise generating a polymer core 22 having a tracer chemical 24 embedded therein; and surrounding the polymer core 22 with an outer layer 26, the outer layer 26 comprising a surfactant 28.

The generating step may comprise interfacial polymerization. The interfacial polymerization may comprise contacting an aqueous layer of fluid with an organic layer of fluid. The tracer chemical 24 may be mixed with the aqueous layer of fluid, or the tracer chemical 24 may be mixed with the organic layer of fluid.

It should be understood that the various embodiments described herein may be utilized in various configurations, without departing from the principles of this disclosure. The embodiments are described merely as examples of useful applications of the principles of the disclosure, which is not limited to any specific details of these embodiments.

The terms “including,” “includes,” “comprising,” “comprises,” and similar terms are used in a non-limiting sense in this specification. For example, if a system, method, apparatus, device, etc., is described as “including” a certain feature or element, the system, method, apparatus, device, etc., can include that feature or element, and can also include other features or elements. Similarly, the term “comprises” is considered to mean “comprises, but is not limited to.”

Of course, a person skilled in the art would, upon a careful consideration of the above description of representative embodiments of the disclosure, readily appreciate that many modifications, additions, substitutions, deletions, and other changes may be made to the specific embodiments, and such changes are contemplated by the principles of this disclosure. Accordingly, the foregoing detailed description is to be clearly understood as being given by way of illustration and example only, the spirit and scope of the invention being limited solely by the appended claims and their equivalents.

What is claimed is:

1. A chemical tracer particle, comprising:
a polymer core;
a tracer chemical embedded in the polymer core; and
an outer layer surrounding the polymer core, the outer layer comprising a surfactant.

2. The chemical tracer particle of claim 1, in which the particle has a maximum outer dimension of less than or equal to 1000 nm.

3. The chemical tracer particle of claim 1, in which the particle is a nanoparticle.

4. The chemical tracer particle of claim 1, in which the polymer core comprises a material selected from the group consisting of polyamide, polyaniline, polyimide, polyurethane, polyurea, polypyrrole, polyester, polysulfonamide, polyphenyl ester and polycarbonate.

5. The chemical tracer particle of claim 1, in which the tracer chemical comprises at least one of the group consisting of halogenated benzene, halogenated aliphatic, halogenated benzoic acid, halogenated benzoic ester, halogenated aldehyde, halogenated ether and halogenated alcohol.

6. A method of tracing flow of well fluid from each of multiple stages in a subterranean well, the method comprising:
injecting into each stage a respective one of multiple chemical tracers, each of the chemical tracers comprising chemical tracer particles, each of the chemical tracer particles comprising a polymer core, a tracer chemical embedded in the polymer core, and an outer layer surrounding the polymer core, the outer layer comprising a surfactant; and
producing the chemical tracers from the stages.

7. The method of claim 6, in which each of the chemical tracers comprises a respective different one of the tracer chemicals.

8. The method of claim 7, in which all of the chemical tracers comprise the same surfactant.

9. The method of claim 7, in which the chemical tracers are adsorbed at a same rate in all of the stages.

10. The method of claim 6, in which each of the chemical tracer particles has a maximum outer dimension of less than or equal to 1000 nm.

11. The method of claim 6, in which the chemical tracer particles comprise nanoparticles.

12. The method of claim 6, in which the polymer core comprises a material selected from the group consisting of polyamide, polyaniline, polyimide, polyurethane, polyurea, polypyrrole, polyester, polysulfonamide, polyphenyl ester and polycarbonate.

13. The method of claim 6, in which the tracer chemicals comprise at least one of the group consisting of halogenated benzene, halogenated aliphatic, halogenated benzoic acid, halogenated benzoic ester, halogenated aldehyde, halogenated ether and halogenated alcohol.

14. A method of producing a chemical tracer particle, the method comprising:
generating a polymer core having a tracer chemical embedded therein; and
surrounding the polymer core with an outer layer, the outer layer comprising a surfactant.

15. The method of claim 14, in which the generating comprises interfacial polymerization.

16. The method of claim 15, in which the interfacial polymerization comprises contacting an aqueous layer of fluid with an organic layer of fluid.

17. The method of claim 16, in which the tracer chemical is mixed with the aqueous layer of fluid.

18. The method of claim 17, in which the tracer chemical is mixed with the organic layer of fluid.

19. The method of claim 14, in which the particle has a maximum outer dimension of less than or equal to 1000 nm.

20. The method of claim 14, in which the particle is a nanoparticle.
